(19) United States
(12) Patent Application Publication
(10) Pub. No.: US 2004/0033218 A1
(43) Pub. Date: Feb. 19, 2004

(54) Use of ecm degrading enzymes for the improvement of cell transplantation

(76) Inventor: Oron Yacoby-Zeevi, Moshav Bizaron 204 (IL)

Correspondence Address: G E Ehrlich, Anthony Castorina, Suite 207, 2001 Jefferson Davis Highway, Arlington, VA, 22202, US

(21) Appl. No.: 10/450,195

(22) PCT Filed: Dec. 17, 2001
(86) PCT No.: PCT/IL01/01169

(51) Int. Cl. A61K 48/00; C12N 5/08

(52) U.S. Cl. 424/93.21; 424/93.7; 435/372

(57) ABSTRACT

Cell preparations which comprise cells carrying an extracellular matrix degrading enzyme and methods of using such cell preparations for improving transplantation efficiency of such cells.

### FIELD AND BACKGROUND OF THE INVENTION

The present invention relates to methods and cell preparations useful in cell and gene therapy.

Cell therapy is a strategy aimed at replacing, repairing, or enhancing the biological function of a damaged tissue or physiological system by means of autologous or allogeneic cell transplantation. There have been major advances in this field in the last few years. Transplantation of stem cells from marrow, blood, or cord blood is now the treatment of choice for a variety of hematological, neoplastic and genetic diseases. Transplantation using less toxic preparative regimens to induce mixed chimerism makes possible application to autoimmune diseases (Thomas E D; Semin. Hematol. 1999, 36(4 Suppl 7):95-103). Cell transplantation depends on the processes of extravasation, migration and invasion.

The Use of Bone Marrow Stromal Cells (BMSCs) for Cell and Gene Therapy:

Bone marrow stromal cells (BMSCs) have the potential to differentiate into a variety of mesenchymal cells. Within the past several years BMSCs have been explored as vehicles for both cell and gene therapy. The cells are relatively easy to isolate from a small aspirates of bone marrow that can be obtained under local anesthesia; they are also relatively easy to expand in culture and are readily transfected with exogenous polynucleotides. Several different strategies are presently being pursued for the therapeutic use of BMSCs:

For example, in the treatment of degenerative arthritis, it was proposed to isolate BMSCs from the bone marrow of a patient having degenerative arthritis, expand the BMSCs in culture, and then use the cells for resurfacing of joint surfaces of the patient by direct injection into the joints. Alternatively, the BMSCs can be implanted into poorly healing bone to enhance the repair process thereof.

In another example, under the umbrella of gene therapy, it was proposed to introduce genes encoding secreted therapeutic proteins, such as insulin, erythropoietin, etc., into the BMSCs derived from the patient and then infuse the cells systemically so that they return to the marrow or other tissues and secrete the therapeutic protein. Additional examples are described herein:

Systemically infused BMSCs, under conditions in which the cells not only repopulate bone marrow, also provide progeny for the repopulating of other tissues such as bone, lung and perhaps cartilage and brain. Recent experiments showed that when donor BMSCs from normal mice are infused in large amounts into young mice that are enfeebled because they express a mutated collagen gene, the normal donor cells replace up to 30% of the cells in bone, cartilage, and brain of the recipient mice. These results were the basis of a clinical trial now in progress for the therapy of bone defects seen in children with sever osteogenesis imperfecta caused by mutations in the genes for type I collagen (Prockop D J; Science 1997, 276: 71-74).

Treatment and potential cure of lysosomal diseases, heretofore considered fatal, has become a reality during the past decade. Bone marrow transplantation, has provided a method for replacement of the disease-causing enzyme deficiency. Cells derived from the donor marrow continue to provide enzyme indefinitely. Several scores of patients with diseases as diverse as metachromatic leukodystrophy, adrenoleukodystrophy, Hurler syndrome (MPS I), Maroteaux-Lamy (MPS VI), Gaucher disease, and fucosidosis have been successfully treated following long term engraftment.

Central nervous system (CNS) manifestations are also prevented or ameliorated in animal models of these diseases following engraftment from normal donors. The microglial cell system has been considered to be the most likely vehicle for enzyme activity following bone marrow engraftment. Microglia in the mature animal or human are derived form the newly engrafted bone marrow. Krivit W et al; Cell Trans. 1995, 4(4): 385-92. In animal models BMSCs can be transfected using retroviruses and can achieve high-level gene expression in vitro and in vivo (Lazarus H M et al; Bone Marrow Transpl. 1995, 16, 557-64).

Because the BMSCs may be capable of extensive proliferation in vitro without loss of pluripotency (in contrast to findings with hematopoietic stem cells), their genetic manipulation and expansion may greatly facilitate gene therapy efforts for hematopoietic disorders.

Marked difficulty in transplanting stromal cells to the bone marrow has been demonstrated; stromal cells transplanted into immunodeficient mice may survive in spleen, liver, or lung but not in bone marrow (Lazarus H M et al; Bone Marrow Transpl. 1995, 16, 557-64).

The Use of CD34+ Progenitor Cells for Cell and Gene Therapies:

The discovery of the severe combined immunodeficiency (scid) mouse mutation has provided a tool for the in vivo analysis of normal and malignant human pluripotent hematopoietic and mesenchymal stem cells. Intravenous injection of irradiated scid mice with human bone marrow, cord blood, or G-CSF cytokine-mobilized peripheral blood mononuclear cells, all rich in human hemopoietic stem cell activity, results in the engraftment of a human hemopoietic system in the murine recipient.

The true functional measure of a long-term renewable stem cell is the capacity to engraft myeloablated recipients, repopulate their hematopoietic systems, and sustain long-term multi-lineage hematopoeisis in vivo. Quantitative analyses of human pluripotent hematopoietic stem cell (HSC) have historically been limited to in vitro assays where the proliferative potential of stem cells is evaluated in the presence of various combinations of cytokines (colony-forming cells in clonal culture, cobblestone area-forming cells, and long-term culture-initiating cells), but these surrogate assays have been shown not to correctly reflect stem cell activity. Over the last 30 years, a number of investigators have attempted to use animals as hosts for quantitative study of the development and differentiation of human pluripotent hematopoietic stem cells. The advantages of animal models, particularly small animal models, are obvious. The development, differentiation, and long-term repopulating capacity of human cells, which can only be determined in vivo, can be ascertained in a small animal model without the need for clinical studies.

This model system should allow detailed identification and characterization of the human pluripotent stem cell and prove readily applicable for in vivo analysis of gene therapy for genetic disorders such as sickle cell anemia and beta-thalassemia which have been studied previously using this model. The extension of the NOD-SCID model to studies of genetic therapy for somatic-based disorders such as adenosine deaminase deficiency has recently been reported and has been shown to provide in vivo information on transduction of stem cells not currently possible using only in vitro methodology. Extension of this model for establishment of hematopoietic chimeras to study transplantation tolerance and for investigation of the stem cell contribution to autoimmunity will provide additional potential avenues for clinical application. Dale L et al; Stem Cells, 1998; 16:166-77.

Hematopoietic stem cells (HSCs) have been defined as being pluripotent (able to give rise to cells of all hemopoietic and lymphoid lineages) and self-renewing (able to give rise to literally billions of progeny cells for essentially a life-time). HSCs can be derived form bone marrow, mobilized peripheral blood, and umbilical cord blood. Cells expressing the CD34 surface antigen constitute a heterogeneous population of hematopoietic cells, including primitive stem cells with self-renewal capacity, and of progenitors committed to myeloid, erythroid and lymphoid development. Large scale devices for the exploitation of CD34+ stem cell selection are now commercially available. In the autologous setting, the primary advantage of using CD34+ selected peripheral blood stem cells (PBSCs) is reduced tumor cell contamination during PBSCs preparation. On the other hand, in the allogeneic setting, CD34+ selection methods are used to reduce the incidence and severity of graft versus host disease (GvHD). Transplantation of autologous selected CD34+ cells from PBSCs gives prompt and stable engraftment. Allogeneic CD34+ selected cells successfully engraft immunomyeloablated recipients through a mega-cell dose effect between HLA-matched pairs. CD34+ selection may also be used as a target of gene therapy, as a source of dendritic cells for cancer immunotherapy and for the treatment of patients with autoimmune diseases (Watanabe T et al; Haematologica 1999, 84(2):167-76). Experience form the transplantation of genetically normal, allogeneic HSCs has demonstrated that a number of genetic diseases of hematopoietic and lymphoid cells can be corrected. Among the disorders that have been successfully treated by allogeneic HSC transplant are hemoglobinopathies, defects of leukocyte production or function, immune deficiencies, lysosomal storage diseases, and stem cell defects, such as Fanconi's anemia. The immunologic limitations of allogeneic bone marrow transplantation (BMT) provide the impetus for consideration of gene therapy using autologous HSCs. Adverse immunologic effects, such as graft rejection, GvHD disease, and the requirements for posttransplant immune suppression could be eliminated. In addition, the availability of techniques to genetically modify HSCs should allow engineering of new, favorable properties into HSCs and their progeny, such as resistance to myelosuppressive effects of chemotherapy or resistance to infection by agents such as HIV-1.

Murine models of lysosomal storage diseases, such as the mucopolysaccharidoses, have been used to demonstrate that either normal congenic bone marrow or gene-corrected autologous bone marrow can provide sufficient levels of the relevant enzyme to ameliorate many of the somatic abnormalities, and have at least a partial benefit on the CNS manifestations.

Although a number of clinical trials have been performed targeting HSC-based diseases, there have been only minimal signs of efficacy, suggesting a failure to transduce reconstituting HSCs. The use of HSCs as the target for correction of genetic diseases may hold an unexpected benefit in that development of immunologic tolerance to the transgene product may be induced. Cytoreductive agents may be administered prior to transplantation of gene-transduced HSCs to prevent unwanted immunologic responses, in addition to the more commonly considered use to “make of space” in the bone marrow for engraftment of the transplanted cells. Newer agents to induce tolerance by blockade of T lymphocyte costimulation may also be applied in the HSC transplantation setting.

Clinically applicable approaches to induce tolerance to the product of genes transferred in HSCs are within reach. Thus the dream of correcting hematopoietic and immune disorders by gene transfer in HSCs, which has been elusive for more than a decade, is slowly becoming a reality (Halene S and Kohn D B; 2000, Hum. Gene Ther. 11: 1259-67).

More than 300 phase I and II gene-based clinical trials have been conducted worldwide for the treatment of cancer and monogenic disorders. Lately, these trials have been extended to the treatment of AIDS and to a lesser extent, cardiovascular diseases. New gene therapy programs to implement procedures of allogeneic tissues or cell transplantation, for neurologic illnesses, autoimmune diseases, allergies and regeneration of tissues are currently in progress. In addition, gene transfer technology is emerging as a powerful tool for innovative vaccine design, which has been termed genetic immunization. Therefore, the potential therapeutic applications of gene transfer technology are enormous. However, the effectiveness of gene therapy programs is still questioned. Furthermore, there is growing concern over the matter of safety of gene delivery and controversy has arisen over the proposal to begin in utero gene therapy clinical trials for the treatment of inherited genetic disorders. The standpoint of the current gene therapy research programs clearly indicates both the presence of a sober optimism among scientists, and a more active role of gene transfer technology in clinical trials for the treatment of cancer, inherited or acquired monogenic disorders, and AIDS. Indeed, gene therapy is one of the fastest growing areas in experimental medicine (Romano G et al; Stem Cells, 2000; 18:19-39).

Dendritic Cells

Dendritic cells (DC) are the most potent antigen presenting cells and the only cells capable of presenting novel antigens to naive T-cells. DCs are professional antigen-presenting cells that are promising adjuvants for clinical immunotherapy. Large numbers of DC can be generated in vitro in the presence of appropriate cytokine cocktails using either adherent peripheral blood mononuclear cells (PBMCs) or CD34+ precursors. DCs, differentiated in vitro, localize preferentially to lymphoid tissue, where they could induce specific immune responses. Thus, these cells have potential implications for immunotherapeutic approaches in the treatment of cancer and other diseases (Mackensen A et al; Cancer Immunol Immunother 1999, 48(2-3):118-22). Three clinical trials have been reported to date that show DC as a promising tool for the immunotherapy of cancer (Esche C et al; Curr Opin Mol Ther. 1999, 1(1):72-81). Efficient genetic modification of CD34+ cell-derived dendritic cells may provide a significant advancement towards the development of immunotherapy protocols for cancer, autoimmune disorders and infectious diseases (Evans J T et al; Gene Ther. 2001, 8(18):1427-35).

Human neoplastic cells are considered to be poorly immunogenic. The development of clinical approaches to the immunotherapy of human tumors thus requires the identification of effective adjuvants. DCs are a specialized system of antigen-presenting cells that could be utilized as natural adjuvants to elicit antitumor immune responses (Di Nicola M et al; Cytokines Cell Mol Ther. 1998, 4(4):265-73).

High-dose chemotherapy with peripheral blood progenitor cell transplantation is a potentially curative treatment option for patients with both hematological malignancies and solid tumors. However, based on a number of clinical studies, there is strong evidence that minimal residual disease (MRD) persists after high-dose chemotherapy in a number of patients, which eventually results in disease recurrence. Therefore, several approaches to the treatment of MRD are currently being evaluated, including treatment with dendritic cell based cancer vaccines and allogeneic adoptive immunotherapy (Brugger W et al; Ann NY Acad Sci. 1999, 872:363-71).

The Use of Peripheral Blood Lymphocytes for Adoptive Immunotherapy:

Adoptive immunotherapy denotes the passive transfer of immunocompetent cells for the treatment of leukemia, cancer, autoimmune or viral diseases. It has regained much interest through the success of treating recurrent leukemia after allogeneic bone marrow transplantation with the transfusion of donor lymphocytes.

Allogeneic bone marrow and hematopoietic progenitor/stem (dentritic cells) cell transplantation has been increasingly used for the treatment of both neoplastic and non-neoplastic disorders. Lymphokine-activated killer (LAK) and tumor-infiltrating lymphocytes (TIL) have been used since the '70s mainly in end-stage patients with solid tumors, but the clinical benefits of these treatments has not been clearly documented. TIL are more specific and potent cytotoxic effectors than LAK, but only in few patients (mainly in those with solid tumors such as melanoma and glioblastoma) can their clinical use be considered potentially useful.

A small subset of peripheral blood natural killer cells (NK), the adhered NK cells (A-NK), has the ability to localize to and induce anti-tumor effects in solid tumor tissues, whereas the majority of circulating non-adhered NK (NA-NK) cells, are not able to do so. NA-NK cells were found to be more cytotoxic than A-NK cells. Thus, both migration into solid tissue and entry of effector cells into a tumor may be related to cellular adhesion molecules expressed on, and to enzymatic activities associated with effector cells. The differences between A-NK and NA-NK cells could be responsible for their different capacities to enter and kill tumor target cells in solid tumor tissues (Vujanovic N L et al; J. Immunol. 1995, 154(1):281-9).

Proteoglycans (PGs):

Proteoglycans (previously named mucopolysaccharides) are remarkably complex molecules and are found in every tissue of the body. They are associated with each other and also with the other major structural components such as collagen and elastin. Some PGs interact with certain adhesive proteins, such as fibronectin and laminin. The long extended nature of the polysaccharide chains of PGs, the glycosaminoglycans (GAGs), and their ability to gel, allow relatively free diffusion of small molecules, but restrict the passage of large macromolecules. Because of their extended structures and the huge macromolecular aggregates they often form, they occupy a large volume of the extracellular matrix relative to proteins (Murry R K and Keeley F W; Biochemistry, Ch. 57. pp. 667-85).

Heparan Sulfate Proteoglycans (HSPGs):

HSPGs are acidic polysaccharide-protein conjugates associated with cell membranes and extracellular matrices. HSPGs bind avidly to a variety of biologic effector molecules, including extracellular matrix components, growth factor, growth factor binding proteins, cytokines, cell adhesion molecules, proteins of lipid metabolism, degradative enzymes, and protease inhibitors. Owing to these interactions, HSPGs play a dynamic role in biology, in fact most functions of the proteoglycans are attributable to the heparan sulfate (HS) chains, contributing to cell-cell interactions and cell growth and differentiation in a number of systems. HS maintains tissue integrity and endothelial cell function. It serves as an adhesion molecule and presents adhesion-inducing cytokines (especially chemokines), facilitating localization and activation of leukocytes. HS modulates the activation and the action of enzymes secreted by inflammatory cells. The function of HS changes during the course of the immune response are due to changes in the metabolism of HS and to the differential expression of and competition between HS-binding molecules. Selvan R S et al; Ann. NY Acad. Sci. 1996, 797: 127-39.

HSPGs are also prominent components of blood vessels (Wight T N et al; Arteriosclerosis, 1989, 9: 1-20). In large vessels HSPGs are concentrated mostly in the intima and inner media, whereas in capillaries HSPGs are found mainly in the subendothelial basement membrane, where they support proliferating and migrating endothelial cells and stabilize the structure of the capillary wall. The ability of HSPGs to interact with extracellular matrix (ECM) macromolecules such as collagen, laminin and fibronectin, and with different attachment sites on plasma membranes suggests a key role for this proteoglycan in the self-assembly and insolubility of ECM components, as well as in cell adhesion and locomotion.

Heparanase—a GAGs Degrading Enzyme:

Degradation of GAGs is carried out by a battery of lysosomal hydrolases. One important enzyme involved in the catabolism of certain GAGs is heparanase. It is an endo-β-glucuronidase that cleaves heparan sulfate at specific interchain sites.

The enzymatic degradation of glycosaminoglycans is reviewed By Ernst et al. (Critical Reviews in Biochemistry and Molecular Biology, 30(5):387-444 (1995). The common feature of GAGs structure is repeated disaccharide units consisting of a uronic acid and hexosamine. Various GAGs differ in the composition of the disaccharide units and in type and level of modifications, such as C5-epimerization and N or O-sulfation. Sulfated GAGs include heparin, heparan sulfate condroitin sulfate, dermatan sulfate and keratan sulfate. Heparan sulfate and heparin are composed of repeated units of glucosamine and glucuronic/iduronic acid, which undergo modifications such as C5-epimerization, N-sulfation and O-sulfation. Heparin is characterized by a higher level of modifications than heparan sulfate.

GAGs can be depolymerized enzymatically either by eliminative cleavage with lyases (EC 4.2.2.-) or by hydrolytic cleavage with hydrolases (EC 3.2.1.-). Often, these enzymes are specific for residues in the polysaccharide chain with certain modifications. GAGs degrading lyases are mainly of bacterial origin. In the eliminative cleavage, C5 hydrogen of uronic acid is abstracted, forming an unsaturated C4-5 bond, whereas in the hydrolytic mechanism a proton is donated to the glycosidic oxygen and creating an O5 oxonium ion followed by water addition which neutralizes the oxonium ion and saturates all carbons (Lindhart et al. 1986, Appl. Biochem. Biotech. 12:135-75). The lyases can only cleave linkages on the non-reducing side of the of uronic acids, as the carboxylic group of uronic acid participates in the reaction. The hydrolyses, on the other hand, can be specific for either of the two bonds in the repeating disaccharides. In pages 414 and 424 of the review, tables 8 and 14, Ernst et al. list the known GAG degrading enzymes. These tables describe substrate specificity, cleavage mechanism, cleavage linkage, product length and mode of action (endo/exolytic). Heparanase is defined as a GAG hydrolase which cleaves heparin and heparan sulfate at the β1,4 linkage between glucuronic acid and glucosamine. Heparanase is an endolytic enzyme and the average product length is 8-12 saccharides. The other known heparin/heparan sulfate degrading enzymes are β-glucuronidase, α-L iduronidase and α-N acetylglucosaminidase which are exolytic enzymes, each one cleaves a specific linkage within the polysaccharide chain and generates disaccharides. In table 8 the authors list two heparanases; platelet heparanase and tumor heparanase, which share the same substrate and mechanism of action. These two were later on found to be identical at the molecular level (Freeman et al. Biochem J. (1999) 342, 361-268, Vlodavsky et al. Nat. Med. 5(7):793-802, 1999, Hullet et al. Nature Medicine 5(7):803-809, 1999).

Heparin and heparan sulfate fragments generated via heparanase catalyzed hydrolysis are inherently characterized by saturated non-reducing ends, derivatives of N-acetyl-glucoseamin. The reducing sugar of heparin or heparan sulfate fragments generated by heparanase hydrolysis contain a hydroxyl group at carbon 4 and it is therefore UV inactive at 232 nm.

Interaction of T and B lymphocytes, platelets, granulocytes, macrophages and mast cells with the subendothelial extracellular matrix (ECM) is associated with degradation of heparan sulfate by heparanase activity. The enzyme is released from intracellular compartments (e.g., lysosomes, specific granules) in response to various activation signals (e.g., thrombin, calcium ionophore, immune complexes, antigens and mitogens), suggesting its regulated involvement in inflammation and cellular immunity. Vlodavsky I et al; Invasion Metas. 1992; 12(2): 112-27. In contrast, various tumor cells appear to express and secrete heparanase in a constitutive manner in correlation with their metastatic potential. Nakajima M et al; J. Cell. Biochem. February 1988; 36(2):157-67. Important processes in the tissue invasion by leukocytes include their adhesion to the luminal surface of the vascular endothelium, their passage through the vascular endothelial cell layer and the subsequent degradation of the underlying basal lamina and extracellular matrix with a battery of secreted and/or cell surface protease and glycosidase activities. Cleavage of HS by heparanase may therefore result in disassembly of the subendothelial ECM and hence may play a decisive role in extravasation of normal and malignant blood-borne cells (Vlodavsky I et al; Inv. Metast. 1992, 12: 112-27, Vlodavsky I et al; Inv. Metast. 1995, 14: 290-302).

It has been previously demonstrated that heparanase may not only function in cell migration and invasion, but may also elicit an indirect neovascular response (Vlodavsky I et al; Trends Biochem. Sci. 1991, 16: 268-71). The ECM HSPGs provide a natural storage depot for bFGF. Heparanase mediated release of active bFGF from its storage within ECM may therefore provide a novel mechanism for induction of neovascularization in normal and pathological situations (Vlodavsky I et al; Cell. Molec. Aspects. 1993, Acad. Press. Inc. pp. 327-343, Thunberg L et al; FEBS Lett. 1980, 117: 203-6). Degradation of heparan sulfate by heparanase results in the release of other heparin-binding growth factors, as well as enzymes and plasma proteins that are sequestered by heparan sulfate in basement membranes, extracellular matrices and cell surfaces. Selvan R S et al; Ann. NY Acad. Sci. 1996, 797: 127-39.

Expression of Heparanase DNA in Animal Cells:

Stably transfected CHO cells expressed the heparanase gene products in a constitutive and stable manner. Several CHO cellular clones have been particularly productive in expressing heparanases, as determined by protein blot analysis and by activity assays. Although the heparanase DNA encodes for a large 543 amino acids protein (expected molecular weight about 65 kDa) the results clearly demonstrate the existence of two proteins, one of about 60-68 kDa and another of about 45-50 kDa. It has been previously shown that a 45-50 kDa protein with heparanase activity was isolated from placenta, Goshen, R. et al. Mol. Human Reprod. 1996, 2: 679-684, and from platelets, Freeman and Parish Biochem. J. 1998, 339:1341-1350. It is thus likely that the 65 kDa protein is the pro-enzyme, which is naturally processed in the host cell to yield the 45 kDa protein. The p50 was found to be active and the p65 protein was not active, further suggesting that the p50 is the active enzyme, and the p65 is a pro-enzyme.

Heparanase Assists in Introducing Biological Material into Patients:

PCT/US00/03353, which is incorporated herein by reference, teaches that when externally added, heparanase adheres to cells. Cells to which heparanase is externally adhered to process the heparanase to an active form. Cells to which an active form of heparanase is externally adhered protect the adhered heparanase from the surrounding medium, such that the adhered heparanase retains its catalytic activity under conditions which otherwise hamper its activity. Cells to which an active form of heparanase is externally adhered, either cells genetically modified to express and extracellularly present or secrete heparanase, or cells to which purified heparanase has been externally added, are much more readily translocatable within the body of experimental animal models, as compared to cells devoid of externally adhered heparanase. Inactive pro-heparanase can be processed by endogenous proteases into its active form, once adhered to cells. Hence, heparanase can be used to assist in introduction of biological materials, such as cells and tissues into desired locations in the bodies of patients.

PCT/IL01/00950 teaches a method of improving embryo transplantation by coating the transplantable embryo with heparanase.

Further details pertaining to heparanase, heparanase gene and their uses can be found in, for example, PCT/US99/09256; PCT/US98/17954; PCT/US99/09255; PCT/US99/25451; PCT/IL00/00358; PCT/US99/15643; PCT/US00/03542; and PCT/US99/06189; and in U.S. Pat. Nos. 6,242,238; 5,968,822; 6,153,187; 6,177,545; and 6,190,875, the contents of which are hereby incorporated by reference.

The efficacy of heparanase in improving cell transplantation was tested in only a very limited number of cases, and it remains to be determined whether, heparanase and other ECM degrading enzymes would assist in cell transplantation in particular cases, such as stem cells, CD34+ progenitor cells, bone marrow stromal cells, dendritic cells and peripheral blood lymphocytes transplantation.

### SUMMARY OF THE INVENTION

According to one aspect of the present invention there is provided a method of improving stem cells transplantation, the method comprising contacting the stem cells, prior to the transplantation with an effective amount of an extracellular matrix degrading enzyme and transplanting the stem cells in a recipient in need thereof.

According to another aspect of the present invention there is provided a stem cells preparation comprising stem cells carrying an exogenous extracellular matrix degrading enzyme.

According to yet another aspect of the present invention there is provided a method of improving CD34+ progenitor cells transplantation, the method comprising contacting the CD34+ progenitor cells, prior to the transplantation with an effective amount of an extracellular matrix degrading enzyme and transplanting the CD34+ progenitor cells in a recipient in need thereof.

According to still another aspect of the present invention there is provided a CD34+ progenitor cells preparation comprising CD34+ progenitor cells carrying an exogenous extracellular matrix degrading enzyme.

According to an additional aspect of the present invention there is provided a method of improving bone marrow stromal cells transplantation, the method comprising contacting the bone marrow stromal cells, prior to the transplantation with an effective amount of an extracellular matrix degrading enzyme and transplanting the bone marrow stromal cells in a recipient in need thereof.

According to yet an additional aspect of the present invention there is provided a bone marrow stromal cells preparation comprising bone marrow stromal cells carrying an exogenous extracellular matrix degrading enzyme.

According to still an additional aspect of the present invention there is provided a method of improving dendritic cells transplantation, the method comprising contacting the dendritic cells, prior to the transplantation with an effective amount of an extracellular matrix degrading enzyme and transplanting the dendritic cells in a recipient in need thereof.

According to a further aspect of the present invention there is provided a dendritic cells preparation comprising dendritic cells carrying an exogenous extracellular matrix degrading enzyme.

According to still a further aspect of the present invention there is provided a method of improving peripheral blood lymphocytes transplantation, the method comprising contacting the peripheral blood lymphocytes, prior to the transplantation with an effective amount of an extracellular matrix degrading enzyme and transplanting the peripheral blood lymphocytes in a recipient in need thereof.

According to yet a further aspect of the present invention there is provided a peripheral blood lymphocyte cells preparation comprising peripheral blood lymphocytes carrying an exogenous extracellular matrix degrading enzyme.

According to further features in preferred embodiments of the invention described below, the cells are of autologous origin.

According to still further features in the described preferred embodiments the cells are of allogeneic origin.

According to still further features in the described preferred embodiments transplanting is effected intravenously, intratracheally, intrauterinally, intraperitoneally, topically or locally.

According to still further features in the described preferred embodiments transplanting is via injection into bone marrow.

According to still further features in the described preferred embodiments the cells are adult derived cells.

According to still further features in the described preferred embodiments the cells are embryo derived cells.

According to still further features in the described preferred embodiments the cells are genetically modified cells.

According to still further features in the described preferred embodiments the extracellular matrix degrading enzyme is selected from the group consisting of a collagenase, a glycosaminoglycans degrading enzyme and an elastase.

According to still further features in the described preferred embodiments the glycosaminoglycans degrading enzyme is selected from the group consisting of a heparanase, a connective tissue activating peptide, a heparinase, a glucoronidase, a heparitinase, a hyluronidase, a sulfatase and a chondroitinase.

According to still further features in the described preferred embodiments, upon contacting, the extracellular matrix degrading enzyme is in an active form.

According to still further features in the described preferred embodiments, upon the contacting, the extracellular matrix degrading enzyme is in an inactive form and is activatable into an active form via a protease.

According to still further features in the described preferred embodiments the extracellular matrix degrading enzyme is heparanase.

According to still further features in the described preferred embodiments the heparanase is a mature heparanase.

According to still further features in the described preferred embodiments the heparanase is a pro-heparanase, cleavable into mature heparanase.

The present invention successfully addresses the shortcomings of the presently known configurations by providing methods and cell preparations which allow improved efficacy of cell transplantation.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show details of the invention in more detail than is necessary for a fundamental understanding of the invention, the description taken with the drawings making apparent to those skilled in the art how the several forms of the invention may be embodied in practice.

In the drawings:

FIG. 1 shows a Western blot analysis demonstrating the fate of heparanase in heparanase coated splenocytes. Heparanase-coated and non-coated splenocytes, 3×10⁵ cells, were subjected to Western blot analysis using anti-p45 heparanase polyclonal antibodies.

FIG. 2 is a survival graph demonstrating mice survival time following adoptive transfer of heparanase-treated allogeneic splenocytes. CB6F1 mice were injected with 2×10⁵ Lewis lung carcinoma cells IV. Four days later the mice were either injected with Hanks solution (Control), or with 10⁷ splenocytes (Splen.), or with 10⁷ heparanase-treated splenocytes (Splen.+Hepa). The survival time of the animals was recorded and expressed in percent of surviving animals at a given time.

FIG. 3 is a graph demonstrating the heparanase activity of 3×10⁵ heparanase-treated (black boxes), and non-treated (empty circles) CD34+ cells. Heparanase activity was analyzed using the radiolabeled ECM assay, by gel filtration. Results are expressed in cpm.

FIG. 4a is a graph demonstrating the effect of heparanase on human stem cells transplantation. NOD-SCID mice were transplanted with heparanase-treated (+) and untreated (−) human CD34+ cells. After 8 weeks the bone marrow of the NOD-SCID mice, was analyzed by flow cytomety using specific FITC-conjugated anti-human CD45 monoclonal antibodies. The human leukocytes in the mouse bone marrow are expressed in percent of human CD45 positive cells.

FIG. 4b is a graph demonstrating the effect of heparanase on the differentiation of transplanted human stem cells. NOD-SCID mice were transplanted with heparanase-treated (+) and untreated (−) human CD34+ cells. After 8 weeks the bone marrow of the NOD-SCID mice, was analyzed by flow cytomety using specific FITC-conjugated anti-human CD15 monoclonal antibodies. The human myeloid cells in the mouse bone marrow are expressed in percent of human CD15 positive cells.

FIG. 5 is a graph demonstrating the effect of heparanase on human CD34+ cells transplantation. NOD-SCID mice were transplanted with heparanase-treated (with hepa) and untreated (w/o hepa) human CD34+ cells. After 6 weeks the bone marrow of the NOD-SCID mice, was analyzed by flow cytomety using specific FITC-conjugated anti-human CD45 monoclonal antibodies. The human leukocytes in the mouse bone marrow are expressed in percent of human CD45 positive cells.

FIG. 6 shows a Western blot analysis demonstrating the fate of heparanase in heparanase coated BMSCs. Heparanase-coated and non-coated BMSCs, 10⁵ cells, were subjected to Western blot analysis using anti-p45 heparanase polyclonal antibodies.

FIG. 7 shows a PCR analysis demonstrating the effect of heparanse on the transplantation of BMSCs. Gamma-irradiated, 3 weeks old Lewis rats were injected intravenously with BMSCs, either treated (lanes 1-6), or not treated (lanes 7-12) with heparanase. After 2 weeks the female acceptor's tissues were snap frozen in liquid nitrogen. DNA was prepared from the livers, lungs, bones, brain, and heart. The DNA was then subjected to PCR using the sry2 primers. The PCR product of the sry gene was about 350 bp.

### DESCRIPTION OF THE PREFERRED EMBODIMENTS

The present invention is of methods and cell preparations which can be used in cell and genetic therapy.

The principles and operation of methods and preparations according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

According to one aspect of the present invention there is provided a method of improving stem cells transplantation, the method comprising contacting the stem cells, prior to the transplantation with an effective amount of an extracellular matrix degrading enzyme and transplanting the stem cells in a recipient in need thereof.

According to another aspect of the present invention there is provided a stem cells preparation comprising stem cells carrying an exogenous extracellular matrix degrading enzyme.

According to yet another aspect of the present invention there is provided a method of improving CD34+ progenitor cells transplantation, the method comprising contacting the CD34+ progenitor cells, prior to the transplantation with an effective amount of an extracellular matrix degrading enzyme and transplanting the CD34+ progenitor cells in a recipient in need thereof.

According to still another aspect of the present invention there is provided a CD34+ progenitor cells preparation comprising CD34+ progenitor cells carrying an exogenous extracellular matrix degrading enzyme.

The stem cells can be adult derived cells. Alternatively, the stem cells can be embryo derived cells.

As used herein, the term “carrying” with respect to an exogenous extracellular matrix degrading enzyme includes loaded, coated, transfected or transformed with the exogenous extracellular matrix degrading enzyme. Methods of transfecting and/or transforming cells ex vivo, so as to induce said cells to express and secrete an extracellular matrix degrading enzyme are well known in the art and are further described in the references listed under the Examples section that follows. By “carrying” it is ment that the total amount of the enzyme is higher than the endogenous amount thereoff, prior to loading, coating, transfecting or transforming.

Improving transplantation efficiency of stem cells, such as CD34+ progenitor cells has therapeutic advantages in the treatment of several diseases, syndromes and/or conditions. In one example, CD34+ progenitor cells implanted as herein described can be used to repopulate a destroyed, compromised or disfunctioning hemopoietic system in a recipient in need thereof, such as a myeloablated recipient, so as to sustain long-term multi-lineage hematopoeisis in vivo. Experience form the transplantation of genetically normal, allogeneic HSCs has demonstrated that a number of genetic diseases of hematopoietic and lymphoid cells can be corrected via stem cells transplantation. Among the disorders that have been successfully treated by allogeneic HSC transplant are hemoglobinopathies, defects of leukocyte production or function, immune deficiencies, lysosomal storage diseases, such as mucopolysaccharidoses, and stem cell defects, such as Fanconi's anemia. In addition, the availability of techniques to genetically modify HSCs will allow engineering of new, favorable properties into HSCs and their progeny, such as resistance to myelosuppressive effects of chemotherapy or resistance to infection by agents such as HIV-1.

According to an additional aspect of the present invention there is provided a method of improving bone marrow stromal cells transplantation, the method comprising contacting the bone marrow stromal cells, prior to the transplantation with an effective amount of an extracellular matrix degrading enzyme and transplanting the bone marrow stromal cells in a recipient in need thereof.

According to yet an additional aspect of the present invention there is provided a bone marrow stromal cells preparation comprising bone marrow stromal cells carrying an exogenous extracellular matrix degrading enzyme.

Improving transplantation efficiency of bone marrow stromal cells (BMSCs) has therapeutic advantages in the treatment of several diseases, syndromes and/or conditions.

Bone marrow stromal cells (BMSCs) have the potential to differentiate into a variety of mesenchymal cells. Within the past several years BMSCs have been explored as vehicles for both cell and gene therapy. The cells are relatively easy to isolate from a small aspirates of bone marrow that can be obtained under local anesthesia; they are also relatively easy to expand in culture and are readily transfected with exogenous polynucleotides. Several different strategies are presently being pursued for the therapeutic use of BMSCs. For example, in the treatment of degenerative arthritis, it was proposed to isolate BMSCs from the bone marrow of a patient having degenerative arthritis, expand the BMSCs in culture, and then use the cells for resurfacing of joint surfaces of the patient by direct injection into the joints. Alternatively, the BMSCs can be implanted into poorly healing bone to enhance the repair process thereof. In another example, under the umbrella of gene therapy, it was proposed to introduce genes encoding secreted therapeutic proteins, such as insulin, erythropoietin, etc., into the BMSCs derived from the patient and then infuse the cells systemically so that they return to the marrow or other tissues and secrete the therapeutic protein. Systemically infused BMSCs, under conditions in which the cells not only repopulate bone marrow, also provide progeny for the repopulation of other tissues such as bone, lung and perhaps cartilage and brain. Recent experiments showed that when donor BMSCs from normal mice are infused in large amounts into young mice that are enfeebled because they express a mutated collagen gene, the normal donor cells replace up to 30% of the cells in bone, cartilage, and brain of the recipient mice. These results were the basis of a clinical trial now in progress for the therapy of bone defects seen in children with sever osteogenesis imperfecta caused by mutations in the genes for type I collagen. Treatment and potential cure of lysosomal diseases, heretofore considered fatal, has become a reality during the past decade. Bone marrow transplantation, has provided a method for replacement of the disease-causing enzyme deficiency. Cells derived from the donor marrow continue to provide enzyme indefinitely. Several scores of patients with diseases as diverse as metachromatic leukodystrophy, adrenoleukodystrophy, Hurler syndrome (MPS I), Maroteaux-Lamy (MPS VI), Gaucher disease, and fucosidosis have been successfully treated following long term engraftment. Central nervous system (CNS) manifestations are also prevented or ameliorated in animal models of these diseases following engraftment from normal donors. The microglial cell system has been considered to be the most likely vehicle for enzyme activity following bone marrow engraftment. Microglia in the mature animal or human are derived form the newly engrafted bone marrow. In animal models BMSCs can be transfected using retroviruses and can achieve high-level gene expression in vitro and in vivo.

According to still an additional aspect of the present invention there is provided a method of improving dendritic cells transplantation, the method comprising contacting the dendritic cells, prior to the transplantation with an effective amount of an extracellular matrix degrading enzyme and transplanting the dendritic cells in a recipient in need thereof.

According to a further aspect of the present invention there is provided a dendritic cells preparation comprising dendritic cells carrying an exogenous extracellular matrix degrading enzyme.

Improving transplantation efficiency of dendritic cells (BMSCs) has therapeutic advantages in the treatment of several diseases, syndromes and/or conditions.

Dendritic cells (DC) are the most potent antigen presenting cells and the only cells capable of presenting novel antigens to naive T-cells. DCs are professional antigen-presenting cells that are promising adjuvants for clinical immunotherapy. Large numbers of DC can be generated in vitro in the presence of appropriate cytokine cocktails using either adherent peripheral blood mononuclear cells (PBMC) or CD34+ precursors. DCs, differentiated in vitro, localize preferentially to lymphoid tissue, where they could induce specific immune responses. Thus, these cells have potential implications for immunotherapeutic approaches in the treatment of cancer and other diseases. Efficient genetic modification of CD34+ cell-derived dendritic cells may provide a significant advancement towards the development of immunotherapy protocols for cancer, autoimmune disorders and infectious diseases. Human neoplastic cells are considered to be poorly immunogenic. The development of clinical approaches to the immunotherapy of human tumors thus requires the identification of effective adjuvants. DCs are a specialized system of antigen-presenting cells that could be utilized as natural adjuvants to elicit antitumor immune responses. High-dose chemotherapy with peripheral blood progenitor cell transplantation is a potentially curative treatment option for patients with both hematological malignancies and solid tumors. However, based on a number of clinical studies, there is strong evidence that minimal residual disease (MRD) persists after high-dose chemotherapy in a number of patients, which eventually results in disease recurrence. Therefore, several approaches to the treatment of MRD are currently being evaluated, including treatment with dendritic cell based cancer vaccines and allogeneic adoptive immunotherapy (which means the passive transfer of allogeneic lymphocytes, including NK cells to a patient).

According to still a further aspect of the present invention there is provided a method of improving peripheral blood lymphocytes transplantation, the method comprising contacting the peripheral blood lymphocytes, prior to the transplantation with an effective amount of an extracellular matrix degrading enzyme and transplanting the peripheral blood lymphocytes in a recipient in need thereof.

According to yet a further aspect of the present invention there is provided a peripheral blood lymphocyte cells preparation comprising peripheral blood lymphocytes carrying an exogenous extracellular matrix degrading enzyme.

The cells used while implementing the methods of the present invention can be of autologous or allogeneic origin. Such cells can be collected from a subject or donor using well established protocols. Such cells can be obtained from peripheral blood, bone marrow and/or cord blood. Such cells are preferably administered to a recipient in need thereof intravenously, intratracheally, intrauterinally, intraperitoneally, topically or locally, or via injection into the bone marrow.

Depending on the medical condition to be treated, the cells according to the present invention can be genetically modified cells. Genetically modified cells are cells that underwent genetic manipulation so as to introduce exogenous polynucleotides into their genome. Such polynucleotides typically include a sequence encoding a protein and regulatory sequences which regulate its expression. Exemplary proteins include hormones, such as insulin and growth hormone, enzymes such as glucocerebrosidase, β-glucoronidase and adenosine deaminase, and other proteins such as β-globin, CFTR, etc. Methods of genetically modifying cells and ex-vivo propagating genetically modified cells are well known in the art and are described, for example, in the citations listed under the Examples section that follows.

It is shown in the Examples section that follows that active heparanase carried by different cell types assists such cells to better extravasate into different body tissues. Heparanase is an extracellular matrix degrading enzyme. It is hence anticipated that other extracellular matrix degrading enzymes, such as collagenases, glycosaminoglycans degrading enzymes, such as connective tissue activating peptide, heparinase, glucoronidase, heparitinase, hyluronidase, sulfatase and chondroitinase, will function in this respect in a way similar to that of heparanase. These enzymes and others are available in an enriched form from various sources. The genes encoding these enzymes have been cloned, such that recombinant enzymes are either available or can be readily made available.

The above enzymes are naturally produced by cells and are thereafter secreted into the extracellular matrix where their exert their enzymatic activity. Such enzymes are typically available in either a mature active form or as proenzymes which are far less or not active. While reducing the present invention to practice, it was uncovered that, once applied ex-vivo to cells, proheparanase is proteolitically cleaved into its active form—mature heparanase.

Hence, while implementing the present invention, the mature, active form or, in the alternative, the proenzyme, inactive form of any of the above extracellular matrix degrading enzymes can be used.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

The Graft Versus Tumor (GVT) Effect of Transferred Allogeneic Heparanase-Treated Immunocompetent Cells

In this example the graft versus tumor (GVT) effect of transferred allogeneic heparanase-treated immunocompetent cells was evaluated.

Materials and Experimental Procedures

Heparanase: CHO-p65 heparanase (1.693 mg/ml; Batch No. 11-1) was used in all experiments performed. CHO-p65 heparanase was prepared according to the protocol described in WO 01/7297. The enzyme was diluted in DMEM+10% FCS, 2 mM Glutamin, 40 μg/ml Gentamycin 1:85 (final heparanase concentration 20 μg/ml).

Cells: Lewis lung carcinoma (D122) derived from a primary tumor were used in this study. These cancer cells were cultured in DMEM growth medium supplemented with 10% FCS, 2% Glutamin, Gentamycin, under 8% CO₂ atmosphere at 37° C. to subconfluency. Splenocytes from Balb/C-nude mice were also used in this study. Splenocytes were cultured in RPMI growth medium supplemented with 10% FCS under 5% CO₂ at atmosphere at 37° C. to 10⁷ cells/ml.

Mice: CB6F1 (7-9 weeks ) and Balb/C-nude (10-12 weeks) male mice from Harlan Laboratories Israel, Ltd. (Rehovot, Israel) were used in this study. The health status of the animal used in this study was examined on arrival. Only animals in good health were acclimatized to experimental conditions. During the study period animals were housed within an animal facility. Animals were kept in groups of maximum 8 mice in polypropylene cages (43×27×18 cm³), and groups of maximum 5 mice in polypropylene cages (29×19×12 cm³), fitted with solid bottoms and filled with wood shavings as bedding material. Animals were provided ad libitum a commercial rodent diet (Harlan Teklad TRM Ra/Mouse Diet) and allowed free access to drinking water, supplied to each cage via polyethylene bottles with stainless steel sipper-tubes. Automatically controlled environmental conditions were set to maintain temperature at 20-24° C. with a relative humidity of 30-70%, a 12-hour light/12-hour dark cycle and sufficient air changes/hour in the study room. CB6F1 male mice were marked using numbered metal earrings. A cage card contained the study name and relevant details as to treatment group. At the end of the study, animals were sacrificed by cervical dislocation.

Experimental metastasis induction: D122 cells, 2×10⁵ cells per 0.2 ml PBS, were injected intravenously, in the tail vein of CB6F1 mice (day 0).

splenocytes preparation: On day 3 splenocytes were prepared according to the following protocol: Spleens from 10 Balb/C-nude mice were obtained in a sterile manner. The cells were squeezed out into sterile PBS using a mesh. The cells were pooled, washed and incubated 5 minutes with erythrocyte lysis buffer (10 times the cells volume) (155 mM NH₄Cl, 10 mM KHCO₃, 0.1 mM EDTA pH-7.3) at 20-25° C. The cells were then washed twice with wash buffer (2 mM EDTA, PBS pH-7.2, 0.5% BSA). The cells were counted (3.6×10⁸ mononuclear cells) and divided into two 75 ml flasks. The cells, 10⁷ cells/ml, were incubated in RPMI (Beit Haemek) +10% FCS (Beit Haemek) +22 nM recombinant mouse IL-2 (R&D) at 37° C. at 5% CO₂ atmosphere for 12 hours. On day 4, one flask containing splenocytes was incubated with 20 μg/ml p65-heparanase for 4 hours at 37° C. under 5% CO₂ atmosphere.

splenocytes injection: On day 4 splenocytes in 0.25 ml Hanks solution was injected intravenously to the CB6F1 mice that were injected with the D122 cells on day 0. Group A was injected with 0.25 ml Hanks solution only, group B was injected with splenocytes and group C was injected with heparanase-treated splenocytes.

Heparanase activity and expression of coated cells: The treated and untreated splenocytes were subjected to the ECM and Western blot analyses, using the protocols described in, for example, U.S. Pat. No. 5,968,822, which is incorporated herein by reference.

Experimental set up: CB6F1 (7-9 weeks) male mice were injected with Lewis lung carcinoma (D122) cells. Consequently, the animals were injected with either hanks solution, or splenocytes (derived from 10-12 weeks Balb/C-nude male mice, treated or not treated with heparanase prior to their intravenous administration so as to test the effect of heparanase on the ability of the splenocytes to prevent tumor development. Two independent experiments were performed in accordance with the following experimental set up:

Experiment No. 1

| Group | Group | | |
|---|---|---|---|
| No | Size | Tumor | Treatment |
| A | n = 7 | Experimental metastasis | Hanks solution |
| | | (2 × 10⁵ D122 cells, IV) | |
| B | n = 7 | Experimental metastasis | Splenocytes, |
| | | (2 × 10⁵ D122 cells, IV) | 10⁷, IV |
| D | n = 7 | Experimental metastasis | Splenocytes + heparanase, |
| | | (2 × 10⁵ D122 cells, IV) | 10⁷, IV |

Since neither of the animal died by day 30, 2 animals of control group A were killed by cervical dislocation, in order to see whether they developed metastases in their lungs. One animal did not have metastases, while the other one had a huge amount of metastases in the lungs. The experiment was therefore continued. Animals that died, or animals which exhibited severe dyspnea or loss of weight were killed by cervical dislocation and their body and lung weights were measured. The experiment terminated on day 56. Two animals of groups B and 2 of group C that were still alive were killed by cervical dislocation and their body and lung weights were measured.

Experiment No. 2

| Group | Group | | |
|---|---|---|---|
| No | Size | Tumor | Treatment |
| A | n = 8 | Experimental metastasis | Hanks solution |
| | | (2 × 10⁵ D122 cells, IV) | |
| B | n = 8 | Experimental metastasis | Splenocytes, |
| | | (2 × 10⁵ D122 cells, IV) | 3 × 10⁶, IV |
| C | n = 8 | Experimental metastasis | Splenocytes, |
| | | (2 × 10⁵ D122 cells, IV) | 15 × 10⁶, IV |
| D | n = 8 | Experimental metastasis | Splenocytes + heparanase, |
| | | (2 × 10⁵ D122 cells, IV) | 15 × 10⁶, IV |
| E | n = 8 | Experimental metastasis | Splenocytes + heparanase, |
| | | (2 × 10⁵ D122 cells, IV) | 3 × 10⁶, IV |

The animal's body weight was measured weekly. When the first animal died on day 17, the experiment terminated and the animals were killed by cervical dislocation. The lungs were excised and their weight was measured. The lungs were observed macroscopically to detect metastases.

In the assessment of metastases in the lungs in Experiment No. 2, 0 indicated the absence of metastases and 1 indicated the presence of metastases in the lungs. The number of animals in the groups treated with heparanase and the groups that were not treated with heparanase was compared.

Experimental Results

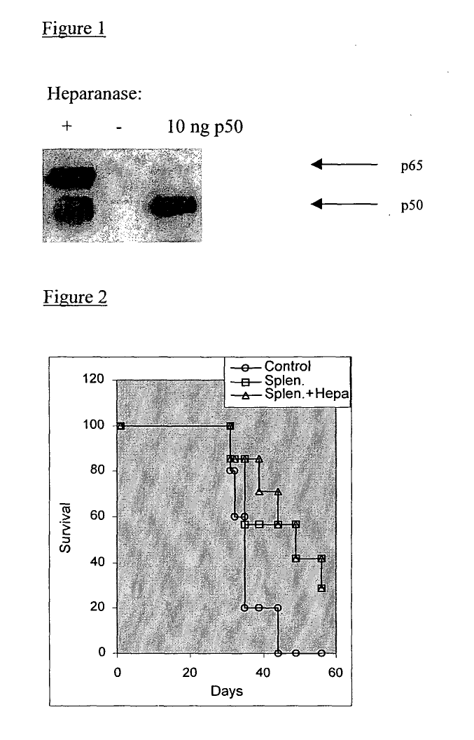

The heparanase-coated splenocytes exhibited p65 and p50 heparanase forms, suggesting that the exogenous p65-heparanase bound to the splenocytes and was processed by them to the p50-active heparanase form (FIG. 1). The heparanase-coated splenocytes possessed high heparanase activity as shown by the DMB assay summarized in Table 1.

TABLE 1
The heparanase activity of splenocytes following
their treatment with heparanase

| | Heparanase activity | |
|---|---|---|
| Heparanase treatment | (O.D.₅₃₀) | Delta O.D.₅₃₀ |
| − | 0.147 | |
| + | 0.225 | 0.078 |

Heparanase-coated and non-coated splenocytes, 1.5 × 10⁶ cells, were subjected to the DMB assay. The activity is expressed by O.D₅₃₀.

Experiment No. 1: All the animals (5/5, two animals were killed on day 30) of group A died by day 44. The animals, 2/7, of the splenocytes-treated groups did not die until the end of the study on day 56. The results are summarized in FIG. 2. One animal of the heparanase-treated group (C) did not have metastases in the lungs. The lungs of the control group (A) and the splenocytes-control group (B) had more and bigger metastases in the lungs when compared to the heparanase-treated splenocytes group (C), which is reflected by the lungs weights. The results are summarized in Table 2.

TABLE 2
The lungs weight (grams) of mice following the adoptive transfer of
heparanase-treated allogeneic splenocytes:

| | | | | Splenocytes + |
|---|---|---|---|---|
| | | Control C | Splenocytes Sp | Heparanase SpH |
| | | 1.535 | 0.659 | 0.341 |
| | | 0.6 | 1.627 | 0.83 |
| | | 2.03 | 1.43 | 0.759 |
| | | 1.924 | 1.87 | 0.25 |
| | | 1.631 | 1.58 | 1.167 |
| Mean | | 1.544 | 1.4332 | 0.6694 |
| SD | | 0.565549 | 0.460787 | 0.375889 |
| Student | SpH - C | | 0.371616 | 0.011907 |
| t-test | SpH - Sp | | | 0.010819 |

CB6F1 mice were injected with 2 × 10⁵ Lewis lung carcinoma cells IV. Four days later the mice were either injected with Hanks solution (Control), or with 10⁷ splenocytes (Sp), or with 10⁷ heparanase-treated splenocytes (SpH). At day of death the lungs were excised and weighed.

Experiment No. 2: The animals, in the control group (A) all developed metastases in the lungs. In the splenocytes-control groups (groups B and C) 15/16 animals developed metastases in the lungs, while only 9/16 animals in the heparanase-treated groups (groups D and E) developed metastases in the lungs (7/16 animals did not develop macroscopic metastases in the lungs). The results are summarized in Table 3.

TABLE 3
The presence of lung metastases and
lungs weight following the adoptive
transfer of heparanase-treated allogeneic splenocytes

| Group & | | Body | Body | | |
|---|---|---|---|---|---|
| treatment | Weight | Weight | Lungs | | |
| (+/− | Animal | (grams) | (grams) | Weight | Presence of |
| heparanase) | Numbers | 11/10 | 29/10 | (grams) | Metastases |
| A− | 451 | 27.6 | 27.4 | 1.329 | + |
| A− | 452 | 24.5 | 24.1 | 0.964 | + |
| A− | 453 | 29.2 | 29.8 | 0.594 | + |
| A− | 454 | 30.1 | 30.0 | 1.130 | + |
| A− | 455 | 26.7 | 23.4 | 1.370 | + |
| A− | 456 | 25.0 | 24.0 | 1.065 | + |
| A− | 457 | 26.4 | 27.7 | 0.852 | + |
| A− | 458 | 25.0 | 21.4 | 0.685 | + |
| B− | 459 | 24.0 | 24.5 | 0.811 | + |
| B− | 460 | 24.7 | 23.0 | 1.488 | + |
| B− | 461 | 27.1 | 25.4 | 1.319 | + |
| B− | 462 | 29.9 | 33.4 | 0.751 | + |
| B− | 463 | 28.7 | 30.6 | 1.049 | + |
| B− | 465 | 26.3 | 23.8 | 1.322 | + |
| B− | 466 | 25.4 | 27.2 | 0.198 | − |
| B− | 467 | 24.2 | 25.8 | 1.028 | + |
| C− | 468 | 24.6 | 23.3 | 1.016 | + |
| C− | 470 | 25.0 | 28.6 | 0.951 | + |
| C− | 471 | 26.8 | 24.7 | 1.411 | + |
| C− | 472 | 28.8 | 32.2 | 0.573 | + |
| C− | 473 | 30.2 | 30.9 | 1.199 | + |
| C− | 474 | 26.2 | 27.5 | 1.105 | + |
| C− | 475 | 31.0 | 30.9 | 1.300 | + |
| C− | 476 | 28.1 | 29.4 | 0.303 | + |
| D+ | 477 | 24.8 | 27.7 | 0.206 | − |
| D+ | 478 | 27.8 | 30.6 | 0.202 | − |
| D+ | 479 | 27.3 | 27.0 | 1.148 | + |
| D+ | 480 | 24.1 | 24.2 | 1.120 | + |
| D+ | 481 | 24.9 | 24.4 | 0.925 | + |
| D+ | 482 | 27.7 | 26.5 | 1.438 | + |
| D+ | 483 | 28.0 | 28.1 | 1.139 | + |
| D+ | 484 | 27.7 | 31.1 | 0.195 | − |
| E+ | 486 | 26.3 | 26.2 | 1.111 | + |
| E+ | 487 | 24.1 | 25.5 | 0.268 | − |
| E+ | 490 | 27.3 | 25.5 | 1.383 | + |
| E+ | 491 | 28.4 | 31.2 | 0.236 | − |
| E+ | 492 | 25.5 | 28.3 | 0.200 | − |
| E+ | 493 | 30.6 | 30.7 | 1.103 | + |
| E+ | 495 | 27.5 | 26.4 | 1.234 | + |
| E+ | 496 | 26.6 | 29.5 | 0.237 | − |

Conclusions

When comparing the number of animals that had metastases in the lungs in the control groups to the number of animals that had metastases in the lungs in the treated groups, the results obtained from experiment No. 2 suggest that there is a significant difference between the control and treated groups, i.e., heparanase treatment prior to implantation substantially improves the GVT effect of immunocompetent cells. When comparing the lungs weight on the day of death in the control groups to the lungs weigh in the treated group, the results obtained from experiment No. 1 suggest that there was a significant difference between the control and treated group, i.e., heparanase treatment prior to implantation substantially improves the GVT effect of immunocompetent cells. There was no significant effect on survival time, perhaps due to the humanitarian fact that animals were sacrificed immediately when seemed suffering, and not necessarily when they were self parishing.

### Example 2

The Effect of Heparanase on Stem Cell Transplantation.

In this example the effect of heparanase on stem cell transplantation was studied.

Materials and Experimental Procedures

Heparanase: CHO-p65 heparanase (1.693 mg/ml; Batch No. 11-1) was used in all experiments performed. CHO-p65 heparanase was prepared according to the protocol described in WO 01/7297. The enzyme was diluted in DMEM+10% FCS, 2 mM Glutamin, 40 μg/ml Gentamycin, 1:85 (final heparanase concentration 20 μg/ml).

Cells: Human cord blood CD34+ progenitor/stem cells were cultured in RPMI growth medium supplemented with 10% FCS under 5% CO₂ atmosphere at 37° C. to a concentration of 10⁶ cells/ml.

Mice: NOD-SCID female mice, two months of age, from Harlan Laboratories Israel, Ltd. (Rehovot, Israel) were used in this study. The health status of the animal used in this study was examined. Only animals in good health were acclimatized to experimental conditions. During the study period animals were housed within an animal facility. Animals were kept in groups of maximum 5 mice in polypropylene cages (29×19×12 cm³), fitted with solid bottoms and filled with wood shavings as bedding material. Animals were provided ad libitum a commercial rodent diet (Harlan Teklad TRM Ra/Mouse Diet) and allowed free access to drinking water, supplied to each cage via polyethylene bottles with stainless steel sipper-tubes. Automatically controlled environmental conditions were set to maintain temperature at 20-24° C. with a relative humidity of 30-70%, a 12-hour light/12-hour dark cycle and sufficient air changes/hour in the study room. A cage card contained the study name and relevant details as to treatment group. At the end of the study, animals were sacrificed by cervical dislocation.

Animal irradiation: On day 0, mice were irradiated with 375 Gy γ-irradiation, at the Radiation Unit of the Weizmann Institute (Rehovot, Israel).

Human cord blood CD34+ cell separation: On day 0, anti-coagulated cord blood samples (6) were received from the hematology department at the Ichilov hospital, Tel-Aviv, Israel. The samples were diluted 1:4 with PBS containing 2 mM EDTA. 35 ml of diluted cell suspension were carefully layered over 15 ml of Ficoll-Paque (Pharmacia), and centrifuged for 35 minutes at 400×g at 20° C. The interphase cells were collected and washed twice in PBS-EDTA (centrifuged for 10 minutes at 200×g at 20° C.). The CD34+ cells were then separated using the “Isolation of CD34 Progenitor Cells Separation Kit” and the MINI-MACS separator, according to the manufacturers protocol (Miltenyi Biotec). A small sample of the cells was then stained with anti CD34-FITC antibodies. The % of CD34+ cells was estimated using FACS (see “FACS analysis”). Only preparations that contained over 75% CD34 cells were used in the experiments.

Human cord blood CD34+ cell coating with heparanase: The separated CD34+ cells were divided into two 35 mm wells. Heparanase, 20 μg/ml final concentration, was added to one of the wells. The cells were incubated for 16 hours at 37° C. under 5% CO₂, in RPMI growth medium supplemented with 10% FBS.

Human cord blood CD34+ cell injection: On day 1, CD34+ cells, 2×10⁵ cells per 0.5 ml RPMI+10% FBS, were injected intravenously via the tail vein to the irradiated SCID-NOD mice (see experimental set-up below).

FACS analysis of murine bone marrow transplanted with human CD34+ cells: Upon study termination, after 6 weeks, mice were killed by cervical dislocation. Tibias and femurs were collected and the bone marrow flushed with 300 μl RPMI. Subsequently, the cells were incubated with various conjugated monoclonal antibodies for 45 minutes at 4° C., washed twice in PBS, and resuspended in 200 μL of PBS. Flow cytometric analysis was performed on the FACS Calibur (Becton Dickinson, San Jose, Calif., USA) and data on 10,000 cells were acquired. The forward scatter threshold was set to permit analysis of viable leukocytes. The monoclonal antibodies used were anti human CD19-APC (Caltag, Burlingam, Calif., USA), anti human CD45-PerCP (Becton Dickinson, Lexington, Ky., USA), anti human CD15-FITC (Caltag, Burlingam, Calif., USA) and anti human CD3-PE (Caltag, Burlingam, Calif., USA).

Heparanase activity and expression of coated cells: The treated and untreated CD34+ cells were subjected to the ECM analysis, using established protocols described in, for example, U.S. Pat. No. 5,968,822, which is incorporated herein by reference.

Experimental Set-Up:

| | | |
|---|---|---|
| Group | Group | |
| No. | Size | Treatment |

Experiment No. 1:

| | | |
|---|---|---|
| C | n = 7 | CD34 + cells |
| H | n = 7 | Heparanase-coated |
| | | CD34 + cells |

Experiment No. 2

| | | |
|---|---|---|
| C | n = 5 | CD34 + cells |
| H | n = 5 | Heparanase-coated |
| | | CD34 + cells |

In Experiment No. 2: One animal of the control group died on day 20.

Statistical analysis: The statistical analysis of the effect of heparanase on CD34+ cells transplantation used the unpaired Students T Test. Since in experiment #2 the % of human cells within the bone marrow of an animal in the treated group (3+) was < than the mean value minus two standard deviations, it was excluded from the statistical analysis.

Experimental Results

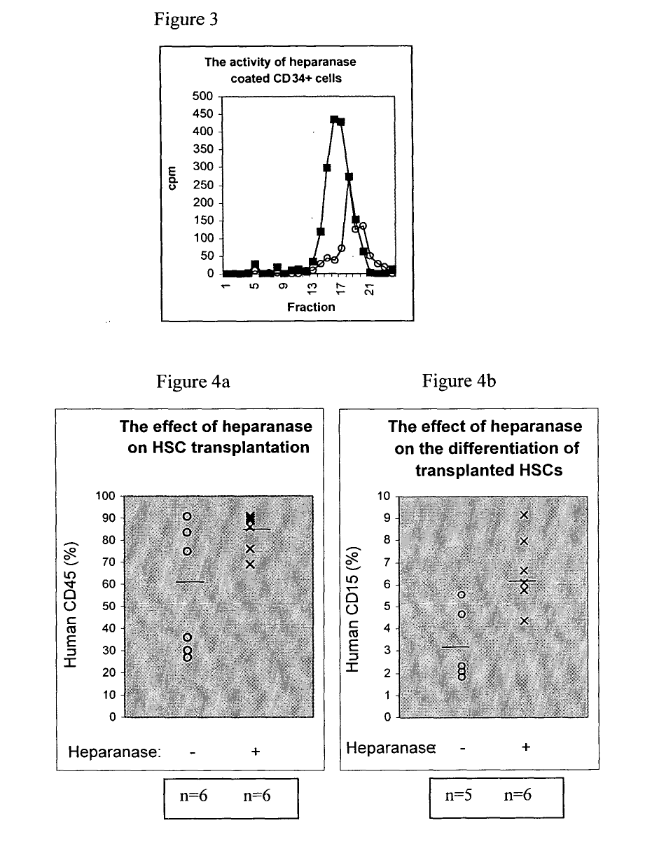

The heparanase-treated CD34+ cells (2×10⁵ cells) expressed high heparanase activity as shown by the ECM assay (FIG. 3).

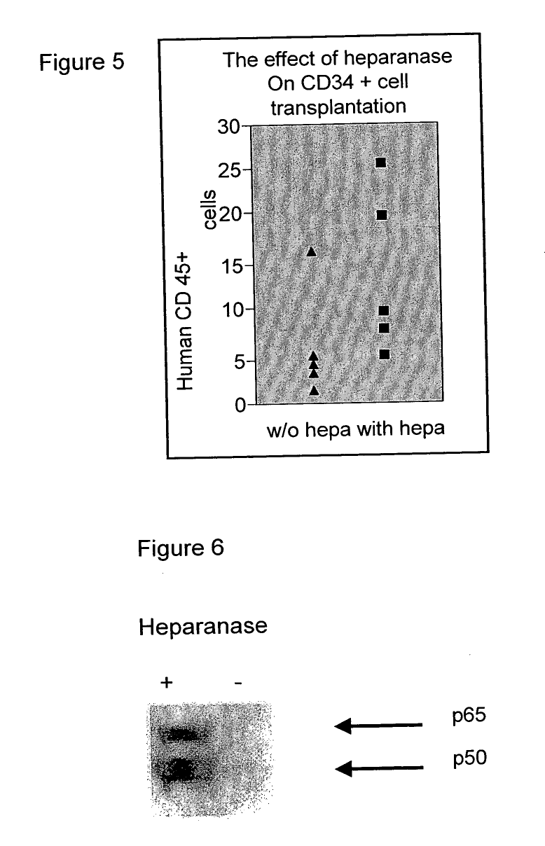

Experiment No. 1: The % of human leukocytes in the mouse bone marrow was analyzed using specific anti-human-CD45 by flow cytometry. The results are summarized in Table 4 and FIG. 5.

TABLE 4

| Heparanase | − | + |
|---|---|---|
| | 0.99 | 5.23 |
| | 3 | 7.42 |
| | 3.94 | 9.29 |
| | 5.31 | 20.2 |
| | 16.35 | 25.61 |
| Mean | 5.918 | 13.55 |
| SD | 6.0396 | 8.8686 |

Experiment No. 2: The % of human leukocytes in the mouse bone marrow was analyzed using specific anti-human-CD45 by flow cytometry. The % of human B-cells, T-cells, and myeloid cells was analyzed using anti-human-CD19, -CD3 and -CD15 respectively. The results are summarized in Table 5 and FIGS. 4a and 4b.

TABLE 5

The effect of heparanase on:

| | CD45 | CD3 | CD15 | CD19 |
|---|---|---|---|---|
| 1− | 30.14 | 2.65 | 1.84 | nd |
| 2− | 35.95 | 2.99 | 2.31 | 42.3 |
| 3− | 26.89 | 3.03 | 2.1 | 41.76 |
| 4− | 83.52 | 5.3 | 5.56 | 36.55 |
| 5− | 90.73 | 19.01 | 21.89* | 38 |
| 6− | 74.88 | 3.38 | 4.66 | 29.44 |
| Mean w/o* | 57.01833 | | 3.294 | |
| SD w/o* | 29.09258 | | 1.696225 | |
| 1+ | 90.8 | 6.81 | 9.14 | 38.33 |
| 2+* | 32.84 | 2.75 | 2.44 | 14.38 |
| 3+ | 89.46 | 5.82 | 7.96 | 34.8 |
| 4+ | 76.06 | 4.35 | 6.1 | 32.09 |
| 5+ | 85.94 | 4.74 | 6.61 | 37.63 |
| 6+ | 69 | 3.17 | 4.35 | 28.91 |
| 7+ | 89.18 | 4.97 | 5.76 | 41.46 |
| Mean w/o* | 83.40667 | | 6.653333 | |
| SD w/o* | 8.860913 | | 1.691291 | |

Conclusions

In Experiment No. 2 heparanase significantly (p<0.04) improved the transplantation of human CD34+ cells in the NOD-SCID mouse model, as reflected by the % of cells in the mouse bone marrow that express the human CD45. In Experiment No. 1 the results are not statistically significant although the trend is obvious. In addition, the % of human cells expressing CD15 in the mouse bone marrow was significantly higher in the heparanase-treated group, suggesting that the transient expression of heparanase induces the differentiation of myeloid cells.

### Example 3

The Effect of Heparanase on the Transplantation of Bone Marrow Stromal Cells in a Rat Model

In this Example the effect of heparanase on bone marrow stromal cells (BMSCs) transplantation was studied.

Heparanase: CHO-p65 heparanase (1.693 mg/ml; Batch No. 11-1) was used in all experiments performed. CHO-p65 heparanase was prepared according to the protocol described in WO 01/7297. The enzyme was diluted in DMEM+10% FCS, 2 mM Glutamin, 40 μg/ml Gentamycin 1:170 (final heparanase concentration 10 μg/ml).

Cells: BMSCs were grown in Low-glucose DMEM growth medium supplemented with 10% FCS, under 8% CO₂ atmosphere at 37° C. to confluency.

Rats: Lewis rats both (3) males (6 weeks old) and (18) females (3 weeks old) from Harlan Laboratories Israel, Ltd. (Rehovot, Israel) were used in this study. The health status of the animal used in this study was examined. Only animals in good health were acclimatized to experimental conditions. The health status of the animal used in this study was examined. Only animals in good health were acclimatized to experimental conditions. During the study period animals were housed within an animal facility. Animals were kept in groups of maximum 5 rats in polypropylene cages (43×27×18 cm³), fitted with solid bottoms and filled with wood shavings as bedding material. Animals were provided ad libitum a commercial rodent diet (Harlan Teklad TRM Ra/Mouse Diet) and allowed free access to drinking water, supplied to each cage via polyethylene bottles with stainless steel sipper-tubes. Automatically controlled environmental conditions were set to maintain temperature at 20-24° C. with a relative humidity of 30-70%, a 12-hour light/12-hour dark cycle and sufficient air changes/hour in the study room. A cage card contained the study name and relevant details as to treatment group. At the end of the study, animals were sacrificed by cervical dislocation.

Animal irradiation: On day 0, rats were irradiated with 450 Gy γ-irradiation, at the Radiation Unit of the Weizmann Institute (Rehovot, Israel).

BMSCs: Femurs and tibias form 2 male, 45 days old, Lewis rats or C57BL mice, were obtained from Harlan Biotech Israel, Ltd. (Rehovot, Israel), in a sterile manner. Bone marrow cells were flushed out, and cultured in low glucose (1 g/L) DMEM?supplemented with 10% FCS (Gibco BRL, Rockville, Md., USA), Gentamycin, 2 mM Glutamine (all purchased from Beit Haemek, Israel). Cultures were maintained in a humidified, 8% CO₂, 37° C., incubator. Following 3 days of incubation, non-adhered cells were washed out, and the adherent cells were re-cultured in the complete DMEM medium. The medium was changed twice a week thereafter.

BMSCs coating with heparanase: When the BMSCs cultures were confluent, some of the cells were incubated with 10 μg/ml p65-heparanase, final concentration, for 3 hours at 37° C. The cells were then trypsinized and counted.

BMSCs injection: On day 1, BMSCs, 3×10⁶ cells per 0.3 ml PBS (see experimental set-up, below), were injected intravenously via the tail vein to the irradiated rats.

DNA extraction from female tissues: Upon study termination, animals were euthenized, and the following organs and tissues were collected: Brain, bone, heart, spleen, lung, liver and bone marrow. Half of each organ was frozen in liquid nitrogen and the remaining of the organ preserved in paraformaldehyde. DNA was extracted from the frozen tissues using the High Pure PCR Template Preparation Kit (Roche Diagnostics, GmbH, Manheim, Germany), according to the manufacturers protocol.

PCR analysis: 250 ng DNA was used for each PCR reaction. PCR program was: 95° C.—5 minutes, 40×[95° C.—1 minute, 62° C.—30 seconds, 72° C.—1 minute], 72° C.—7 minutes. The following primers were used:

| | |
|---|---|
| sry2R: | |
| 5′-AGG CAA CTT CAC GCT GCA AAG TA-3′ | (SEQ ID NO:1) |
| Sry2F: | |
| 5′-AGC TTT CGG ACG AGT GAC AGT TG-3′ | (SEQ ID NO:2) |
| β-actinR: | |
| 5′-AGG CAG CTC ATA GCT CTT CTC-3′ | (SEQ ID NO:3) |
| β-actinF: | |
| 5′-GAT CAT GTT TGA GAC CTT CAA C-3′ | (SEQ ID NO:4) |
| sry1R: | |
| 5′-CTT CAG TCT CTG CGC CTC CT-3′ | (SEQ ID NO:5) |
| sry1F: | |
| 5′-GGA GAG AGG CAC AAG TTG GC-3′ | (SEQ ID NO:6) |

Heparanase activity and expression of coated cells: The treated and untreated cells were subjected to the DMB and Western blot analyses, using the protocols described in, for example, U.S. Pat. No. 6,190,875, which is incorporated herein by reference.

Experimental set-up:

| Group | Group | |
|---|---|---|
| No. | Size | Treatment |
| C | n = 9 | BMSCs |
| H | n = 9 | Heparanase-coated BMSCs |

On day 4 an animal from the treated group (H), which had a tail wound, died. On day 13 and 14, 3 animals from group C and 2 animals from group H, died. Since, day 14, all animals of group C exhibited tachypnea, piloerection, tearing and apathy, the study was terminated, the rats were euthenized by intraperitoneal injection of nembutal, and post mortem analysis was performed.

Experimental Results

Macroscopic observations (icteric liver, pale spleen, yellow bone marrow, inflammed lungs, and pale membranes) suggested that the animals suffered from irradiation damage, and that the treated group (H) were less affected.

The BMSCs cells (10⁵ cells) bound the p65-heparanase and processed it to its active p50 form as shown by Western blot analysis (FIG. 6). The cells expressed high heparanase activity as shown by the DMB assay (Table 6).

TABLE 6

| | Heparanase activity | |
|---|---|---|
| Heparanase treatment | (O.D.₅₃₀) | Delta O.D₅₃₀ |
| − | 0.4183 | |
| + | 0.7291 | 0.3108 |

The heparanase activity of BMSCs following their treatment with heparanase was analyzed using the DMB assay and is expressed in O.D.₅₃₀.

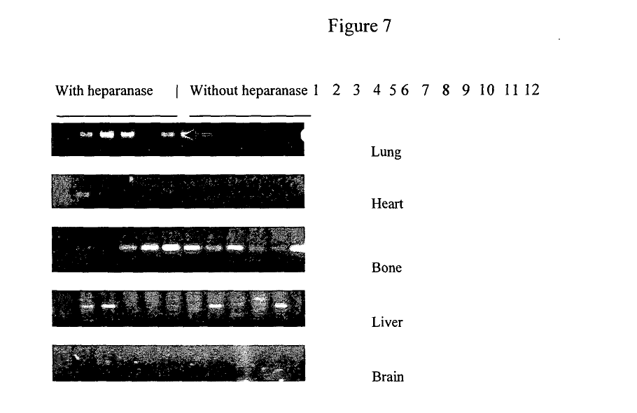

The expression of the male specific (y chromosome) sry gene within the tissues of the recipient females was analyzed using PCR (FIG. 7). The sry gene was present in the lungs of 4/6 animals in the treated group (BMSCs+hearanase, group H) and in the lungs of 1/6 animals in the control group (BMSCs, group C). The number of animals exhibiting the sry gene in the liver and bone was similar in both groups. The sry gene was expressed in 3 and 1 BMSCs+hearanase-treated animals, in the heart and brain, respectively, whereas it was not expressed in any of the control animals. The heparnase was not expressed in the bone-marrow and spleen of either group. The amount of DNA from each animal that was used for the PCR reaction was compared by the PCR analysis of the samples using the β-actin primers, and was found similar (not shown).

Conclusions

Heparanase improves BMSCs transplantation, mainly into the lungs of irradiated rats.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of improving stem cells transplantation, the method comprising contacting the stem cells, prior to said transplantation with an effective amount of an extracellular matrix degrading enzyme and transplanting said stem cells in a recipient in need thereof.

2. The method of claim 1, wherein said stem cells are of autologous origin.

3. The method of claim 1, wherein said stem cells are of allogeneic origin.

4. The method of claim 1, wherein said transplanting is effected intravenously, intratracheally, intrauterinally, intraperitoneally, topically or locally.

5. The method of claim 1, wherein said transplanting is via injection into bone marrow.

6. The method of claim 1, wherein said stem cells are adult derived stem cells.

7. The method of claim 1, wherein said stem cells are embryo derived stem cells.

8. The method of claim 1, wherein said stem cells are genetically modified stem cells.

9. The method of claim 1, wherein said extracellular matrix degrading enzyme is selected from the group consisting of a collagenase, a glycosaminoglycans degrading enzyme and an elastase.

10. The method of claim 9, wherein said glycosaminoglycans degrading enzyme is selected from the group consisting of a heparanase, ???, a heparinase, a glucoronidase, a heparitinase, a hyluronidase, a sulfatase and a chondroitinase.

11. The method of claim 1, wherein, upon said contacting, said extracellular matrix degrading enzyme is in an active form.

12. The method of claim 1, wherein, upon said contacting, said extracellular matrix degrading enzyme is in an inactive form or is activatable into an active form by the cells.

13. The method of claim 1, wherein said extracellular matrix degrading enzyme is heparanase.

14. The method of claim 13, wherein said heparanase is a mature heparanase.

15. The method of claim 13, wherein said heparanase is a pro-heparanase, cleavable into mature active heparanase.

16. A stem cells preparation comprising stem cells carrying an exogenous extracellular matrix degrading enzyme.

17. The stem cells preparation of claim 16, wherein said stem cells are of autologous origin.

18. The stem cells preparation of claim 16, wherein said stem cells are of allogeneic origin.

19. The stem cells preparation of claim 16, wherein said stem cells are adult derived stem cells.

20. The stem cells preparation of claim 16, wherein said stem cells are embryo derived stem cells.

21. The stem cells preparation of claim 16, wherein said stem cells are genetically modified stem cells.

22. The stem cells preparation of claim 16, wherein said extracellular matrix degrading enzyme is selected from the group consisting of a collagenase, a glycosaminoglycans degrading enzyme and an elastase.

23. The stem cells preparation of claim 22, wherein said glycosaminoglycans degrading enzyme is selected from the group consisting of a heparanase, a connective tissue activating peptide, a heparinase, a glucoronidase, a heparitinase, a hyluronidase, a sulfatase and a chondroitinase.

24. The stem cells preparation of claim 16, wherein, upon said pre-contact, said extracellular matrix degrading enzyme is in an active form.

25. The stem cells preparation of claim 16, wherein, upon said pre-contact, said extracellular matrix degrading enzyme is in an inactive form and is activatable into an active form via a protease.

26. The stem cells preparation of claim 16, wherein said extracellular matrix degrading enzyme is heparanase.

27. The stem cells preparation of claim 26, wherein said heparanase is a mature heparanase.

28. The stem cells preparation of claim 26, wherein said heparanase is a pro-heparanase, cleavable into a mature heparanase.

29. A method of improving CD34+ progenitor cells transplantation, the method comprising contacting the CD34+ progenitor cells, prior to said transplantation with an effective amount of an extracellular matrix degrading enzyme and transplanting said CD34+ progenitor cells in a recipient in need thereof.

30. The method of claim 29, wherein said CD34+ progenitor cells are of autologous origin.

31. The method of claim 29, wherein said CD34+ progenitor cells are of allogeneic origin.

32. The method of claim 29, wherein said transplanting is effected intravenously, intratracheally, intrauterinally, intraperitoneally, topically or locally.

33. The method of claim 29, wherein said transplanting is via injection into bone marrow.

34. The method of claim 29, wherein said CD34+ progenitor cells are from bone marrow, peripheral blood or cord blood.

35. The method of claim 29, wherein said CD34+ progenitor cells are genetically modified CD34+ progenitor cells.

36. The method of claim 29, wherein said extracellular matrix degrading enzyme is selected from the group consisting of a collagenase, a glycosaminoglycans degrading enzyme and an elastase.

37. The method of claim 36, wherein said glycosaminoglycans degrading enzyme is selected from the group consisting of a heparanase, a connective tissue activating peptide, a heparinase, a glucoronidase, a heparitinase, a hyluronidase, a sulfatase and a chondroitinase.

38. The method of claim 29, wherein, upon said contacting, said extracellular matrix degrading enzyme is in an active form.

39. The method of claim 29, wherein, upon said contacting, said extracellular matrix degrading enzyme is in an inactive form and is activatable into an active form via a protease.

40. The method of claim 29, wherein said extracellular matrix degrading enzyme is heparanase.

41. The method of claim 40, wherein said heparanase is a mature heparanase.

42. The method of claim 40, wherein said heparanase is a pro-heparanase, cleavable into mature heparanase.

43. A CD34+ progenitor cells preparation comprising CD34+ progenitor cells carrying an exogenous extracellular matrix degrading enzyme.

44. The CD34+ progenitor cells preparation of claim 43, wherein said CD34+ progenitor cells are of autologous origin.

45. The CD34+ progenitor cells preparation of claim 43, wherein said CD34+ progenitor cells are of allogeneic origin.

46. The CD34+ progenitor cells preparation of claim 43, wherein said CD34+ progenitor cells are from bone marrow, peripheral blood or cord blood.

47. The CD34+ progenitor cells preparation of claim 43, wherein said CD34+ progenitor cells are genetically modified CD34+ progenitor cells.

48. The CD34+ progenitor cells preparation of claim 43, wherein said extracellular matrix degrading enzyme is selected from the group consisting of a collagenase, a glycosaminoglycans degrading enzyme and an elastase.

49. The CD34+ progenitor cells preparation of claim 48, wherein said glycosaminoglycans degrading enzyme is selected from the group consisting of a heparanase, a connective tissue activating peptide, a heparinase, a glucoronidase, a heparitinase, a hyluronidase, a sulfatase and a chondroitinase.

50. The CD34+ progenitor cells preparation of claim 43, wherein, upon said pre-contact, said extracellular matrix degrading enzyme is in an active form.

51. The CD34+ progenitor cells preparation of claim 43, wherein, upon said pre-contact, said extracellular matrix degrading enzyme is in an inactive form and is activatable into an active form via a protease.

52. The CD34+ progenitor cells preparation of claim 43, wherein said extracellular matrix degrading enzyme is heparanase.

53. The CD34+ progenitor cells preparation of claim 52, wherein said heparanase is a mature heparanase.

54. The CD34+ progenitor cells preparation of claim 52, wherein said heparanase is a pro-heparanase, cleavable into a mature heparanase.

55. A method of improving bone marrow stromal cells transplantation, the method comprising contacting the bone marrow stromal cells, prior to said transplantation with an effective amount of an extracellular matrix degrading enzyme and transplanting said bone marrow stromal cells in a recipient in need thereof.

56. The method of claim 55, wherein said bone marrow stromal cells are of autologous origin.

57. The method of claim 55, wherein said bone marrow stromal cells are of allogeneic origin.

58. The method of claim 55, wherein said transplanting is effected intravenously, intratracheally, intrauterinally, intraperitoneally, topically or locally.

59. The method of claim 55, wherein said transplanting is via injection into bone marrow.

60. The method of claim 55, wherein said bone marrow stromal cells are from bone marrow, peripheral blood or cord blood.

61. The method of claim 55, wherein said bone marrow stromal cells are genetically modified bone marrow stromal cells.

62. The method of claim 55, wherein said extracellular matrix degrading enzyme is selected from the group consisting of a collagenase, a glycosaminoglycans degrading enzyme and an elastase.

63. The method of claim 62, wherein said glycosaminoglycans degrading enzyme is selected from the group consisting of a heparanase, a connective tissue activating peptide, a heparinase, a glucoronidase, a heparitinase, a hyluronidase, a sulfatase and a chondroitinase.

64. The method of claim 55, wherein, upon said contacting, said extracellular matrix degrading enzyme is in an active form.

65. The method of claim 55, wherein, upon said contacting, said extracellular matrix degrading enzyme is in an inactive form and is activatable into an active form via a protease.

66. The method of claim 55, wherein said extracellular matrix degrading enzyme is heparanase.

67. The method of claim 66, wherein said heparanase is a mature heparanase.

68. The method of claim 66, wherein said heparanase is a pro-heparanase, cleavable into mature heparanase.

69. A bone marrow stromal cells preparation comprising bone marrow stromal cells carrying an exogenous extracellular matrix degrading enzyme.

70. The bone marrow stromal cells preparation of claim 69, wherein said bone marrow stromal cells are of autologous origin.

71. The bone marrow stromal cells preparation of claim 69, wherein said bone marrow stromal cells are of allogeneic origin.

72. The bone marrow stromal cells preparation of claim 69, wherein said bone marrow stromal cells are from bone marrow, peripheral blood or cord blood.

73. The bone marrow stromal cells preparation of claim 69, wherein said bone marrow stromal cells are genetically modified bone marrow stromal cells.

74. The bone marrow stromal cells preparation of claim 69, wherein said extracellular matrix degrading enzyme is selected from the group consisting of a collagenase, a glycosaminoglycans degrading enzyme and an elastase.

75. The bone marrow stromal cells preparation of claim 74, wherein said glycosaminoglycans degrading enzyme is selected from the group consisting of a heparanase, a connective tissue activating peptide, a heparinase, a glucoronidase, a heparitinase, a hyluronidase, a sulfatase and a chondroitinase.

76. The bone marrow stromal cells preparation of claim 69, wherein, upon said pre-contact, said extracellular matrix degrading enzyme is in an active form.

77. The bone marrow stromal cells preparation of claim 69, wherein, upon said pre-contact, said extracellular matrix degrading enzyme is in an inactive form and is activatable into an active form via a protease.

78. The bone marrow stromal cells preparation of claim 69, wherein said extracellular matrix degrading enzyme is heparanase.

79. The bone marrow stromal cells preparation of claim 78, wherein said heparanase is a mature heparanase.

80. The bone marrow stromal cells preparation of claim 78, wherein said heparanase is a pro-heparanase, cleavable into a mature heparanase.

81. A method of improving dendritic cells transplantation, the method comprising contacting the dendritic cells, prior to said transplantation with an effective amount of an extracellular matrix degrading enzyme and transplanting said dendritic cells in a recipient in need thereof.

82. The method of claim 81, wherein said dendritic cells are of autologous origin.

83. The method of claim 81, wherein said dendritic cells are of allogeneic origin.

84. The method of claim 81, wherein said transplanting is effected intravenously, intratracheally, intrauterinally, intraperitoneally, topically or locally.

85. The method of claim 81, wherein said transplanting is via injection into bone marrow.

86. The method of claim 81, wherein said dendritic cells are from bone marrow, peripheral blood or cord blood.

87. The method of claim 81, wherein said dendritic cells are genetically modified dendritic cells.

88. The method of claim 81, wherein said extracellular matrix degrading enzyme is selected from the group consisting of a collagenase, a glycosaminoglycans degrading enzyme and an elastase.

89. The method of claim 88, wherein said glycosaminoglycans degrading enzyme is selected from the group consisting of a heparanase, a connective tissue activating peptide, a heparinase, a glucoronidase, a heparitinase, a hyluronidase, a sulfatase and a chondroitinase.

90. The method of claim 81, wherein, upon said contacting, said extracellular matrix degrading enzyme is in an active form.

91. The method of claim 81, wherein, upon said contacting, said extracellular matrix degrading enzyme is in an inactive form and is activatable into an active form via a protease.

92. The method of claim 81, wherein said extracellular matrix degrading enzyme is heparanase.

93. The method of claim 92, wherein said heparanase is a mature heparanase.

94. The method of claim 92, wherein said heparanase is a pro-heparanase, cleavable into mature heparanase.

95. A dendritic cells preparation comprising dendritic cells carrying an exogenous extracellular matrix degrading enzyme.

96. The dendritic cells preparation of claim 95, wherein said dendritic cells are of autologous origin.

97. The dendritic cells preparation of claim 95, wherein said dendritic cells are of allogeneic origin.

98. The dendritic cells of claim 95, wherein said dendritic cells are from bone marrow, peripheral blood or cord blood.

99. The dendritic cells preparation of claim 95, wherein said dendritic cells are genetically modified dendritic cells.

100. The dendritic cells preparation of claim 95, wherein said extracellular matrix degrading enzyme is selected from the group consisting of a collagenase, a glycosaminoglycans degrading enzyme and an elastase.

101. The dendritic cells preparation of claim 100, wherein said glycosaminoglycans degrading enzyme is selected from the group consisting of a heparanase, a connective tissue activating peptide, a heparinase, a glucoronidase, a heparitinase, a hyluronidase, a sulfatase and a chondroitinase.

102. The dendritic cells preparation of claim 95, wherein, upon said pre-contact, said extracellular matrix degrading enzyme is in an active form.

103. The dendritic cells preparation of claim 95, wherein, upon said pre-contact, said extracellular matrix degrading enzyme is in an inactive form and is activatable into an active form via a protease.

104. The dendritic cells preparation of claim 95, wherein said extracellular matrix degrading enzyme is heparanase.

105. The dendritic cells preparation of claim 104, wherein said heparanase is a mature heparanase.

106. The dendritic cells preparation of claim 104, wherein said heparanase is a pro-heparanase, cleavable into a mature heparanase.

107. A method of improving peripheral blood lymphocyte cells transplantation, the method comprising contacting the peripheral blood lymphocyte cells, prior to said transplantation with an effective amount of an extracellular matrix degrading enzyme and transplanting said peripheral blood lymphocyte cells in a recipient in need thereof.

108. The method of claim 107, wherein said peripheral blood lymphocyte cells are of autologous origin.

109. The method of claim 107, wherein said peripheral blood lymphocyte cells are of allogeneic origin.

110. The method of claim 107, wherein said transplanting is effected intravenously, intratracheally, intrauterinally, intraperitoneally, topically or locally.

111. The method of claim 107, wherein said transplanting is via injection into bone marrow.

112. The method of claim 107, wherein said peripheral blood lymphocyte cells are from bone marrow, peripheral blood or cord blood.

113. The method of claim 107, wherein said peripheral blood lymphocyte cells are genetically modified peripheral blood lymphocyte cells.

114. The method of claim 107, wherein said extracellular matrix degrading enzyme is selected from the group consisting of a collagenase, a glycosaminoglycans degrading enzyme and an elastase.

115. The method of claim 114, wherein said glycosaminoglycans degrading enzyme is selected from the group consisting of a heparanase, a connective tissue activating peptide, a heparinase, a glucoronidase, a heparitinase, a hyluronidase, a sulfatase and a chondroitinase.

116. The method of claim 107, wherein, upon said contacting, said extracellular matrix degrading enzyme is in an active form.

117. The method of claim 107, wherein, upon said contacting, said extracellular matrix degrading enzyme is in an inactive form and is activatable into an active form via a protease.

118. The method of claim 107, wherein said extracellular matrix degrading enzyme is heparanase.

119. The method of claim 118, wherein said heparanase is a mature heparanase.

120. The method of claim 118, wherein said heparanase is a pro-heparanase, cleavable into mature heparanase.

121. A peripheral blood lymphocyte cells preparation comprising peripheral blood lymphocyte cells carrying an exogenous extracellular matrix degrading enzyme.

122. The peripheral blood lymphocyte cells preparation of claim 121, wherein said peripheral blood lymphocyte cells are of autologous origin.

123. The peripheral blood lymphocyte cells preparation of claim 121, wherein said peripheral blood lymphocyte cells are of allogeneic origin.

124. The peripheral blood lymphocyte cells preparation of claim 121, wherein said peripheral blood lymphocyte cells are from bone marrow, peripheral blood or cord blood.

125. The peripheral blood lymphocyte cells preparation of claim 121, wherein said peripheral blood lymphocyte cells are genetically modified peripheral blood lymphocyte cells.

126. The peripheral blood lymphocyte cells preparation of claim 121, wherein said extracellular matrix degrading enzyme is selected from the group consisting of a collagenase, a glycosaminoglycans degrading enzyme and an elastase.

127. The peripheral blood lymphocyte cells preparation of claim 126, wherein said glycosaminoglycans degrading enzyme is selected from the group consisting of a heparanase, a connective tissue activating peptide, a heparinase, a glucoronidase, a heparitinase, a hyluronidase, a sulfatase and a chondroitinase.

128. The peripheral blood lymphocyte cells preparation of claim 121, wherein, upon said pre-contact, said extracellular matrix degrading enzyme is in an active form.

129. The peripheral blood lymphocyte cells preparation of claim 121, wherein, upon said pre-contact, said extracellular matrix degrading enzyme is in an inactive form and is activatable into an active form via a protease.

130. The peripheral blood lymphocyte cells preparation of claim 121, wherein said extracellular matrix degrading enzyme is heparanase.

131. The peripheral blood lymphocyte cells preparation of claim 130, wherein said heparanase is a mature heparanase.

132. The peripheral blood lymphocyte cells preparation of claim 130, wherein said heparanase is a pro-heparanase, cleavable into a mature heparanase.
